(19) United States
(12) Patent Application Publication
(10) Pub. No.: US 2012/0252831 A1
(43) Pub. Date: Oct. 4, 2012

(54) COMPOSITIONS OF OPIOID ANTAGONISTS AND METHODS FOR TREATING SCLERODERMA THEREWITH

(75) Inventors: Ibrahim Mustafa Iskender Pisak, Istanbul (TR); Semra Bingol, Istanbul (TR); Mehmet Levent Selamoglu, Istanbul (TR); Mehmet Pisak, Istanbul (TR)

(73) Assignee: Mustafa Nevzat Ilac Sanayii A.S., Istanbul (TR)

(21) Appl. No.: 13/076,590

(22) Filed: Mar. 31, 2011

(51) Int. Cl. A61K 31/485; A61P 19/00

(52) U.S. Cl. 514/282; 514/289

(57) ABSTRACT

Provided are compositions comprising opioid antagonists, such as naltrexone naloxone, or nalmefene, or their pharmaceutically acceptable salts, and methods for treating scleroderma, including systemic sclerosis, therewith.

### FIELD OF THE INVENTION

The invention relates to compositions comprising opioid antagonists, such as naltrexone, naloxone, or nalmefene, or their pharmaceutically acceptable salts, and methods for treating scleroderma, including systemic sclerosis, therewith.

### BACKGROUND OF THE INVENTION

Naltrexone has the chemical name morphinan-6-one, 17-(cyclopropylmethyl)-4,5-epoxy-3,14-dihydroxy-(5α). The molecular formula of naltrexone is C₂₀H₂₃NO₄ and its molecular weight is 341.41 in the anhydrous form (<1% maximum water content). The chemical structure of naltrexone is shown below.

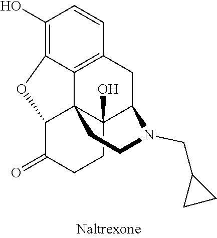

Naltrexone has been approved for use in the treatment of alcoholism or narcotic addiction. It is believed that naltrexone functions by blocking the brain receptors that trigger the effects of alcohol or narcotics. Naltrexone is marketed by Durmed in the form of a tablet under the tradename ReVia® and by Alkermes in the form of a powder for injectable suspension under the tradename Vivitrol®.

Naloxone has the chemical name (−)-17-allyl-4,5α-epoxy-3,14-dihydroxymorphinan-6-one and the molecular formula C₁₉H₂₁NO₄. The chemical structure of naloxone is shown below.

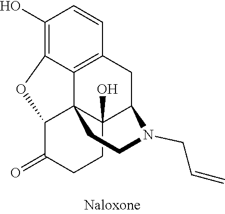

Naloxone is typically administered intravenously because of its short duration of action, and is generally administered to a patient in order to reverse opioid depression, including respiratory depression, induced by natural and synthetic opioids.

Nalmefene has the chemical name 17-cyclopropylmethyl-4,5α-epoxy-6-methylenemorphinan-3,14-diol and the molecular formula C₂₁H₂₅NO₃. The chemical structure of nalmefene is shown below.

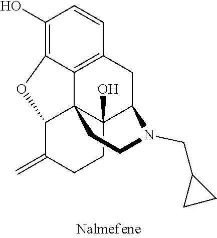

Nalmefene is typically used in the management of alcohol dependence, and also has been investigated for the treatment of other addictions such as pathological gambling and addiction to shopping. Advantages of nalmefene relative to naltrexone include longer half-life, greater oral bioavailability and no observed dose-dependent liver toxicity. As with other drugs of this type, nalmefene can precipitate acute withdrawal symptoms in patients who are dependent on opioid drugs, or more rarely when used post-operatively to counteract the effects of strong opioids used in surgery.

In the early 1980s, it was reported that the administration of low dose naltrexone (less than 10 mg naltrexone per day) increases the production of endogenous endomorphins, especially the endogenous pentapeptide metenkephalin, and increases the number and density of metenkephalin receptors by intermittently blocking opiate receptors. (I. S. Zagon & P. J. McLaughlin, “Naltrexone modulates tumor response in mice with neuroblastoma”, Science, 221: 671-3 (12 Aug. 1983). This increase in metenkephalin is believed to enhance homeostatic regulation of the natural immune function of the human body.

In view of Zagon's findings, Bernard Bihari reported the use of low dose naltrexone for the treatment of patients with AIDS (U.S. Pat. No. 4,888,346) and herpes (U.S. Pat. No. 5,356,900). Further, Nicholas Plotnikoff reported the use of low dose naltrexone for the treatment of herpes, HIV infection, cytomegalovirus, coronavirus, influenza A and Japanese encephalitis. (E. A. Moore & S. Wilkinson, THE PROMISE OF Low DOSE NALTREXONE THERAPY: POTENTIAL BENEFITS IN CANCER, AUTOIMMUNE, NEUROLOGICAL AND INFECTIOUS DISORDERS (McFarland & Company, Inc., Publishers, 2009)).

Scleroderma is a rare disease that affects approximately 19 out of every 1 million people. The cause of scleroderma is unknown, but it is believed that there may be a genetic predisposition for the disease. Genetic abnormalities that have been observed in people diagnosed with scleroderma involve autoimmunity and alteration of endothelial cell and fibroblast function. Indeed, systemic sclerosis is probably the most severe of the auto-immune diseases with 50% mortality within 5 years of diagnosis (Silman A. J. (1991) Mortality from scleroderma in England and Wales 1968-1975. Ann. Rheu. Dis. 50, 95-96).

Scleroderma has a spectrum of manifestations and a variety of therapeutic implications. Types of scleroderma include localized scleroderma, systemic sclerosis, scleroderma-like disorders, and Sine scleroderma (Smith, Textbook of the Autoimmune Diseases, Edited by Lahita, Chiorazzi and Reeves, Lippincott Williams & Wilkins, Philadelphia 2000).

While localized scleroderma is a rare dermatologic disease associated with fibrosis and manifestations limited to skin, systemic sclerosis is a multisystem disease with variable risk for internal organ involvement and variation in the extent of skin disease. Systemic sclerosis can be diffuse or limited. Limited systemic sclerosis is also called CREST (calcinosis, Raynaud's esophageal dysfunction, sclerodaytyl), telangiectasiae). Scleroderma-like disorders are believed to be related to industrial environment exposure. In Sine disease, there is internal organ involvement without skin changes.

The major manifestations of scleroderma and in particular of systemic sclerosis are inappropriate excessive collagen synthesis and deposition, endothelial dysfunction, spasm, collapse and obliteration by fibrosis. Several underlying biological processes are implicated in the initiation, severity and progression of the disease and include vascular dysfunction, endothelial cell activation and damage, leukocyte accumulation, auto-antibody production and crucially, an uncontrolled fibrotic response which may lead to death. (Clements P. J. and Furst D. E. (1996) “Systemic Sclerosis” Williams and Williams, Baltimore).

Fibroblasts have a pivotal role in the pathogenesis of this disease. Primary fibroblasts obtained from patients with scleroderma exhibit many of the characteristic properties of the disease seen in vivo, notably increased extracellular matrix synthesis and deposition, notably of collagen and fibronectin, and altered growth factor and cytokine production such as of transforming growth factor (TGF) and connective tissue growth factor (CTGF) (Strehlow D. and Korn J (1998) Biology of the scleroderma fibroblast. Curr. Opin. Rheumatol. 10, 572-578; LeRoy E. C. (1974) Increased collagen synthesis by scleroderma skin fibroblasts in vitro. J. Clin. Invest. 54, 880-889). There is no curative treatment of scleroderma. Innovative but high-risk therapy proposed autologous stem cell transplantation. (Martini, Maccado, Ravelli et al., Arthritis Rheum. 1999, 42, 807-811).

### SUMMARY OF THE INVENTION

In one embodiment, the invention encompasses methods for treating scleroderma comprising administering to a subject in need thereof an effective amount of an opioid antagonist or a pharmaceutically acceptable salt thereof.

In another embodiment, the invention encompasses kits comprising a unit dose of an opioid antagonist and a label or printed instructions instructing the administration of the opioid antagonist to treat scleroderma.

### DETAILED DESCRIPTION OF THE INVENTION

The present invention provides methods for treating scleroderma comprising administering to a subject in need thereof an effective amount of an opioid antagonist or a pharmaceutically acceptable salt thereof. In one embodiment, the opioid antagonist or pharmaceutically acceptable salt thereof is topically administered to the subject.

As used herein, an “effective amount” is an amount effective for treating scleroderma.

As used herein, the term “treating” scleroderma in a subject refers to subjecting the subject to a pharmaceutical treatment, e.g., the administration of one or more agents, such that at least one symptom of the condition is decreased or prevented from worsening.

In one embodiment, the scleroderma is localized scleroderma, systemic sclerosis, a scleroderma-like disorder, or Sine scleroderma. In some embodiments, symptoms of scleroderma to be treated include inappropriate excessive collagen synthesis and deposition, endothelial dysfunction, spasm, collapse and obliteration by fibrosis.

Suitable opioid antagonists include compounds that block one or more opioid receptors. In some embodiments, the opioid antagonist selectively blocks the mu (μ) opioid receptor, the delta (δ) opioid receptor, or the kappa (κ) opioid receptor. In other embodiments, the opioid antagonist is non-selective. Examples of opioid antagonists include, but are not limited to, naltrexone, naloxone, nalorphine, levallorphan, nalmefene, cyprodime, naltindole, and norbinaltorphimine. In one embodiment, the opioid antagonist is naltrexone. In another embodiment, the opioid antagonist is naloxone. In another embodiment, the opiod antagonist is nalmefene.

In some embodiments, the opioid antagonist is a compound that may exist in the form of one or more stereoisomers, wherein one or more of those steroisomers is therapeutically active. In some embodiments, the opioid antagonist comprises a therapeutically active stereoisomer that is substantially free of other stereoisomers. In other embodiments, the opioid antagonist comprises a therapeutically active stereoisomer that has less than about 50%, less than about 40%, less than about 30%, less than about 20%, less than about 10%, less than about 5%, or less than about 1% by weight of other steroisomers.

The opioid antagonist is typically administered to the subject in the form of a composition for topical or oral administration. In some embodiments, the composition comprises an effective amount of the opioid antagonist and at least one pharmaceutically acceptable excipient.

Compositions for Topical Administration

In some embodiments, the opioid antagonist is administered to the subject in the form of a composition for topical administration.

In some embodiments, the opioid antagonist is present in an amount of about 0.1% to about 5% by weight of the composition. In other embodiments, the opioid antagonist is present in an amount of about 0.5% to about 4%, about 0.5% to about 2%, about 0.5% to about 1.5%, about 1% to about 4%, about 1% to about 3%, about 1% to about 2%, about 2% to about 3%, about 2% to about 4%, about 3% to about 4%, or about 1% by weight of the composition.

The pharmaceutically acceptable excipient may be any topically acceptable non-transdermally effective excipient known by those skilled in the art. Suitable excipients include, but are not limited to, emulsifying agents, stiffening agents, rheology modifiers or thickeners, surfactants, emollients, preservatives, humectants, alkalizing or buffering agents, and solvents.

Suitable emulsifying agents include, but are not limited to cetyl alcohol, cetostearyl alcohol, stearyl alcohol, carboxypolymethylene, polycarbophil, polyethylene glycol, and sorbitan esters. Suitable stiffening agents include, but are not limited to stearyl alcohol, cetostearyl alcohol, and cetyl alcohol. Suitable rheology modifiers or thickeners include, but are not limited to, carbomers such as Carbopol®, and polyoxyethylene tallow amines such as Ethomeen®. Suitable surfactants include, but are not limited to, anionic, cationic, amphoteric, and nonionic surfactants. In some embodiments, the surfactant is sodium lauryl sulfate, cetostearyl alcohol, cetyl alcohol, magnesium lauryl sulfate, a wax, or a combination thereof. Suitable emollients include, but are not limited to, white petrolatum (while vaseline), liquid petrolatum (liquid vaseline), paraffin, or aquaphor. Suitable preservatives include, but are not limited to, antimicrobial preservatives such as nipagin (methyl hydroxybenzoate), nipasol (hydroxybenzoate), butylparaben, ethylparaben, methylparaben, propyl paraben potassium, and propyl paraben sodium. Suitable humectants include, but are not limited to, propylene glycol and propylene glycol alginate. Suitable alkalizing or buffering agents include, but are not limited to, sodium hydroxide and potassium hydroxide. Suitable solvents include, but are not limited to, water.

The composition may be in the form of a gel, cream, ointment, liquid, suspension, solution, emulsion, foam, aerosol or the like for topical administration. Typically, the composition is administered to the subject by spreading (e.g., gel, cream, or ointment) or spraying (e.g., liquid or aerosol) onto the affected area of the skin.

In one embodiment, the composition is in the form of a cream. Typically, the cream comprises an opioid antagonist and one or more of an emulsifying agent, a stiffening agent, a surfactant, an emollient, a preservative, a humectant, an alkalizing or buffering agent, and a solvent. In some embodiments, the cream has a formulation according to Table 1a, 1b, or 1c.

TABLE 1a
Illustrative Cream Formulation

| | Amount (% by weight |
|---|---|
| Ingredient | of the composition) |
| Opioid antagonist | about 0.1%-5% |
| Emulsifying agent | about 2%-5% |
| Stiffening agent | about 1%-45% |
| Surfactant | about 0.5%-2.5% |
| Preservative | about 0.01%-0.6% |
| Humectant | about 1%-15% |
| Alkalizing or | about 0.01%-3% |
| buffering agent | |
| Emollient | about 1%-50% |
| Solvent | q.s |

TABLE 1b
Illustrative Cream Formulation

| | Amount (% by weight |
|---|---|
| Ingredient | of the composition) |
| Opioid antagonist | about 0.5%-2% |
| Emulsifying agent | about 2%-5% |
| Stiffening agent | about 2%-5% |
| Surfactant | about 0.5%-1.5% |
| Preservative | about 0.01%-0.6% |
| Humectant | about 2%-10% |
| Alkalizing or | about 0.01%-3% |
| buffering agent | |
| Emollient | about 15%-30% |
| Solvent | q.s |

TABLE 1c
Illustrative Cream Formulation

| | Amount (% by weight |
|---|---|
| Ingredient | of the composition) |
| Opioid antagonist | about 0.5%-2% |
| Cetyl alcohol and/or | about 2%-5% |
| carboxypolymethylene | |
| Stearyl alcohol | about 2%-5% |
| Sodium lauryl sulfate | about 0.5%-1.5% |
| Nipagin and/or Nipasol | about 0.01%-0.6% |
| Propylene glycol | about 2%-10% |
| Sodium hydroxide | about 0.01%-3% |
| White vaseline and/or | about 15%-30% |
| liquid vaseline | |
| Water | q.s |

In one embodiment, the composition is in the form of an ointment. Typically, the ointment comprises an opioid antagonist and one or more of an emulsifying agent, an emollient, and a preservative. In some embodiments, the ointment has a formulation according to Table 2a, 2b, or 2c.

TABLE 2a
Illustrative Ointment Formulation

| | Amount (% by weight |
|---|---|
| Ingredient | of the composition) |
| Opioid antagonist | about 0.1%-5% |
| Emulsifying agent | about 1%-10% |
| Preservative | about 0.01%-0.6% |
| Emollient | q.s. |

TABLE 2b
Illustrative Ointment Formulation

| | Amount (% by weight |
|---|---|
| Ingredient | of the composition) |
| Opioid antagonist | about 0.5%-2% |
| Emulsifying agent | about 2%-5% |
| Preservative | about 0.01%-0.6% |
| Emollient | q.s. |

TABLE 2c
Illustrative Ointment Formulation

| | Amount (% by weight |
|---|---|
| Ingredient | of the composition) |
| Opioid antagonist | about 0.5%-2% |
| Polyoxyethylene 20 | about 2%-5% |
| sorbitan monooleate | |
| Nipagin and/or Nipasol | about 0.01%-0.6% |
| White vaseline and/or | q.s. |
| liquid vaseline | |

In one embodiment, the composition is in the form of a gel. Typically, the gel comprises an opioid antagonist and one or more of a rheology modifier or thickener, an alkalizing or buffering agent, and a solvent. In some embodiments, the gel has a formulation according to Table 3a, 3b, or 3c.

TABLE 3a
Illustrative Gel Formulation

| | Amount (% by weight |
|---|---|
| Ingredient | of the composition) |
| Opioid antagonist | about 0.1%-5% |
| Rheology modifier or thickener | about 0.5%-2% |
| Alkalizing or buffering agent | about 0.5%-10% |
| Solvent | q.s. |

TABLE 3b
Illustrative Gel Formulation

| | Amount (% by weight |
|---|---|
| Ingredient | of the composition) |
| Opioid antagonist | about 0.5%-2% |
| Rheology modifier or thickener | about 1%-2% |
| Alkalizing or buffering agent | about 0.5%-5% |
| Solvent | q.s |

TABLE 3c
Illustrative Gel Formulation

| | Amount (% by weight |
|---|---|
| Ingredient | of the composition) |
| Opioid antagonist | about 0.5%-2% |
| Carbomer | about 1%-2% |
| Sodium hydroxide | about 0.5%-5% |
| Water | q.s |

Typically, the opioid antagonist is administered to the subject in a total daily dose of up to about 150 mg/cm² of skin. In some embodiments, the opioid antagonist is administered to the subject in a total daily dose of about 5 mg/cm² of skin to about 150 mg/cm² of skin, about 10 mg/cm² of skin to about 100 mg/cm² of skin, about 20 mg/cm² of skin to about 90 mg/cm² of skin, about 30 mg/cm² of skin to about 80 mg/cm² of skin, about 40 mg/cm² of skin to about 70 mg/cm² of skin, or about 50 mg/cm² of skin or about 60 mg/cm² of skin. The total daily dose may be delivered once per day, or divided between multiple doses. In some embodiments, the opioid antagonist is administered 1, 2, 3, 4, or 5 times per day.

Compositions for Oral Administration

In some embodiments, the opioid antagonist is administered to the subject in the form of a composition for oral administration. The oral composition may be in the form of a tablet, capsule, caplet, granule, powder, lozenge, troche, dragee, sachet, cachet, liquid, solution, suspension, emulsion, elixir, or syrup for oral administration. In one embodiment, the oral composition is in the form of a tablet or capsule. The tablet may be in the form of an uncoated tablet, coated tablet (for example with sugar or an enteric coating), effervescent tablet, dispersible tablet, orally-dissolving tablet, or sublingual tablet.

In one embodiment, the oral composition comprises an effective amount of an opioid antagonist and at least one pharmaceutically acceptable excipient. Suitable pharmaceutically acceptable excipients for the above solid oral compositions include, but are not limited to, starches, sugars, diluents, granulating agents, lubricants, binders, disintegrating agents and the like. Suitable pharmaceutically acceptable excipients for the above liquid oral compositions include, but are not limited to, water, glycols, oils, alcohols, flavoring agents, preservatives, coloring agents and the like.

Typically, the opioid antagonist is present in the oral composition in an amount of about 0.1 mg to about 10 mg, about 0.5 mg to about 8 mg, about 1 mg to about 6 mg, or about 1 mg to about 4.5 mg. In one embodiment, the opioid antagonist is present in the oral composition in an amount of about 2 mg.

The topical and oral compositions described above may be administered to the subject daily, every other day, three times a week, twice a week, once a week, or at other appropriate intervals. In some embodiments, the composition is administered until there is complete healing of the scleroderma in the affected area.

The present invention may use lower doses of opioid antagonist than the doses conventionally used for oral administration in the treatment of alcohol or narcotic addiction. The opioid antagonist is administered to the individual in an amount effective to treat the scleroderma. In certain embodiments, the exact dose of opioid antagonist depends upon, by way of non-limiting example, the form in which the opioid antagonist is administered, the subject to be treated, the age, body weight and/or height of the subject to be treated, the preference and experience of the attending physician, the specific opioid antagonist used, the characteristics of the patient, and/or the nature of the condition for which the treatment is sought. Thus, in some embodiments, the dosage of opioid antagonist administered may vary from those disclosed herein. In various embodiments, these factors are determined by those of skill in the medical and pharmaceutical arts in view of the present disclosure.

The methods of the invention may further comprise administration of one or more additional agents effective to treat the scleroderma. The additional agent may include, but not be limited to interferon, in particular interferon-α, a Tumor Necrosis Factor (TNF) antagonist, in particular TNF binding protein I and/or II (TBP I and/or TBP II), or an anti-scleroderma agent selected from the group consisting of angiotensin-converting enzyme (ACE) inhibitors, calcium channel blockers, proton pump inhibitors, non-steroidal anti-inflammatory agents (NSAIDs), cyclooxygenase (COX) inhibitors, corticosteroids, tetracycline, pentoxifylline, bucillamine, geranylgeranyl transferase inhibitors, rotterlin, prolyl-4-hydroxlase inhibitors, c-proteinase inhibitors, lysyl-oxidase inhibitors, relaxin, halofuginone, prostaglandins, prostacyclins, endothelin-I, nitric oxide, angiotensin II inhibitors and anti-oxidants. The additional agent and the opioid antagonist may be administered concurrently or separately. When administered concurrently, the additional agent and the opioid antagonist may be administered in the same or separate compositions.

In another embodiment, the invention encompasses kits comprising a unit dose of opioid antagonist. In one embodiment the unit dose is within a container, which can be sterile, containing an effective amount of opioid antagonist and a pharmaceutically acceptable excipient. The kits can further comprise a label or printed instructions instructing the use of the opioid antagonist to treat scleroderma. The kits can further comprise a device that is useful for administering the unit dose as described herein. Examples of such a device include, but are not limited to, a wand, a dropper, a cotton swab, a pad, or the like.

Having described the invention with reference to certain embodiments, other embodiments will become apparent to one skilled in the art from consideration of the specification. The invention is further defined by reference to the following examples. It will be apparent to those skilled in the art that many modifications, both to materials and methods, may be practiced without departing from the scope of the invention.

### EXAMPLES

### Example 1

### Treatment of Systemic Sclerosis with Naltrexone

Formulations

100 g of naltrexone 1% cream was prepared by mixing 1 g of naltrexone hydrochloride in 99 g of a cream base containing the following excipients: cetyl alcohol, stearyl alcohol, sodium lauryl sulfate, vaseline, nipagin, nipasol, carboxypolymethylene, propyleneglycol, sodium hydroxide and distilled water.

Naltrexone capsules having the following formulation were also prepared.

TABLE 4
Naltrexone Capsule

| Ingredient | Content | |
|---|---|---|
| Naltrexone hydrochloride | 2.00 | mg |
| Magnesium stearate | 0.18 | mg |
| Microcristalline cellulose | q.s. 90.00 | mg |

| | |
|---|---|
| Gelatin capsule No 4 | 1 |

Treatment

A man in his 60s with manifestations of systemic sclerosis including inappropriate excessive collagen synthesis and deposition, endothelial dysfunction, spasms and fibrosis, was treated with the 1% naltrexone cream and 2 mg naltrexone capsules described above.

The naltrexone cream was administered to the affected area of skin three times daily (morning, noon, and evening). The hardening of the skin mostly on the hands, likely caused by collagen synthesis and fibrosis was prevented and became less and less bothersome to the subject, after the use of the cream for 1 month on the hands.

At the same time the cream was being administered, the patient was also administered the naltrexone capsule once daily before bedtime to treat the remaining symptoms. After 3 weeks of treatment, the patient started to breathe much better, most likely as a result of the minimization of the fibrosis in the lungs.

### Example 2

### Naltrexone Topical Formulations

The following are illustrative naltrexone formulations according to the present invention.

TABLE 5
Illustrative Naltrexone Cream Formulation

| | Amount (% by weight |
|---|---|
| Ingredient | of the composition) |
| Naltrexone | 1% |
| Cetyl alcohol | 3.6% |
| Stearyl alcohol | 3.6% |
| Sodium lauryl sulfate | 0.8% |
| Nipagin | 0.1% |
| Nipasol | 0.05% |
| Carboxylpolymethylene | 0.2% |
| Propylene glycol | 5% |
| Sodium hydroxide | 0.03% |
| White vaseline | 13.5% |
| Liquid vaseline | 5.4% |
| Water | q.s |

TABLE 6
Illustrative Naltrexone Ointment Formulation

| | Amount (% by weight |
|---|---|
| Ingredient | of the composition) |
| Naltrexone | 1% |
| Polyoxyethylene 20 | 4% |
| sorbitan monooleate | |
| Nipagin | 0.18% |
| Nipasol | 0.02% |
| White vaseline | 10% |
| Liquid vaseline | q.s. |

TABLE 7
Illustrative Naltrexone Gel Formulation

| | Amount (% by weight |
|---|---|
| Ingredient | of the composition) |
| Naltrexone | 1% |
| Carbomer | 2% |
| Sodium hydroxide | 1.25% |
| Water | q.s |

We claim:

1. A method for treating scleroderma comprising administering to a subject in need thereof an effective amount of an opioid antagonist or a pharmaceutically acceptable salt thereof.

2. The method of claim 1, wherein the opioid antagonist is naltrexone, naloxone, nalorphine, levallorphan, nalmefene, cyprodime, naltindole, or norbinaltorphimine.

3. The method of claim 1, wherein the opioid antagonist is naloxone.

4. The method of claim 1, wherein the opioid antagonist is nalmefene.

5. The method of claim 1, wherein the opioid antagonist is naltrexone.

6. The method of claim 5, wherein the naltrexone has less than about 10% by weight of other steroisomers.

7. The method of claim 5, wherein the naltrexone has less than about 5% by weight of other steroisomers.

8. The method of claim 1, wherein the scleroderma is systemic sclerosis.

9. The method of claim 1, wherein the opioid antagonist is administered in the form of a composition comprising the opioid antagonist and at least one pharmaceutically acceptable excipient.

10. The method of claim 9, wherein the composition is formulated for topical administration.

11. The method of claim 10, wherein the opioid antagonist is present in an amount of about 0.1% to about 5% by weight of the composition.

12. The method of claim 10, wherein the opioid antagonist is present in an amount of about 0.5% to about 2% by weight of the composition.

13. The method of claim 10, wherein the opioid antagonist is present in an amount of about 1% by weight of the composition.

14. The method of claim 10, wherein the composition is in the form of a gel, cream, ointment, liquid, suspension, solution, emulsion, foam, or aerosol.

15. The method of claim 10, wherein the composition is administered to the subject by spreading or spraying the composition onto the affected area.

16. The method of claim 10, wherein the opioid antagonist is administered in a total daily dose of up to about 150 mg/cm² of skin.

17. The method of claim 10, wherein the opioid antagonist is administered in a total daily dose of about 10 mg/cm² to about 100 mg/cm² of skin.

18. The method of claim 10, wherein the opioid antagonist is administered in a total daily dose of about 50 mg/cm² of skin.

19. The method of claim 9, wherein the composition is formulated for oral administration.

20. The method of claim 19, wherein the formulation is in the form of a tablet or capsule.

21. The method of claim 19, wherein the opioid antagonist is present in an amount of about 0.1 mg to about 10 mg.

22. The method of claim 19, wherein the opioid antagonist is present in an amount of about 1 mg to about 4.5 mg.

23. The method of claim 19, wherein the opioid antagonist is present in an amount of about 2 mg.

24. The method of claim 1, wherein the opioid antagonist is administered 1, 2, 3, 4, or 5 times daily.

25. The method of claim 1, further comprising administering to the subject one or more additional agents effective to treat the scleroderma.

26. A kit comprising a unit dose of an opioid antagonist and a label or printed instructions instructing the administration of the opioid antagonist to treat scleroderma.

27. The kit of claim 26, wherein the scleroderma is systemic sclerosis.

28. The kit of claim 26, wherein the administration is topical administration.
